(19) United States
(12) Patent Application Publication
(10) Pub. No.: US 2026/0345554 A1
(43) Pub. Date: Nov. 5, 2026

(54) METHOD FOR TREATING HEARING LOSS AND IMPROVING AUDITORY DISCRIMINATION

(71) Applicant: City University of Hong Kong, Kowloon (HK)

(72) Inventors: Jufang He, Hong Kong (HK); Peipei Zhou, Hong Kong (HK); Xi Chen, Hong Kong (HK)

(21) Appl. No.: 19/188,256

(22) Filed: Apr. 24, 2025

(51) Int. Cl. A61K 31/4045; A61N 7/00; A61P 27/16

(52) U.S. Cl. CPC A61K 31/4045; A61N 7/00; A61P 27/16; A61N 2007/0021; A61N 2007/0073

(57) ABSTRACT

Method for improving neuroplasticity in the auditory cortex in a subject in need thereof includes administrating a CCK-B receptor agonist to the subject. Method for treating hearing loss in a subject in need thereof includes administrating a CCK-B receptor agonist to the subject. Method for improving auditory discrimination ability in a subject in need thereof includes administrating a CCK-B receptor agonist to the subject, which benefits to patients who have auditory processing disorders

### FIELD OF THE INVENTION

This application relates to a method for treating hearing loss and improving auditory discrimination.

### BACKGROUND

Hearing-related disease is a significant global health problem, affecting both the ageing population and an increasing number of younger people due to factors such as environmental noise and unsafe listening practices. The impact of hearing loss extends beyond the auditory system, potentially affecting mental health and cognitive functions. In recent years, research on the mechanism of hearing loss, drugs and methods to prevent hearing loss have made rapid progress. However, there are no pharmacological therapeutics currently in widespread use.

Therefore, there is a need for new therapies for hearing-related diseases, such as auditory processing disorders and hearing loss.

### SUMMARY OF THE INVENTION

An embodiment of this application relates to a method for enabling/improving neuroplasticity in the auditory cortex in a subject in need thereof, which includes administrating a CCK-B receptor agonist to the subject.

Without intending to be bound by theory, it is believed that the ability of CCK-B receptor agonist such as HT-267 to cross the BBB and promote neuroplasticity in the auditory cortex as disclosed herein has not been reported before. This invention shows that HT-267 can directly influence central auditory processing, which is crucial for improving hearing sensitivity and auditory discrimination. Without intending to be bound by theory, it is believed that CCK-B receptor agonists can facilitate neuroplasticity, which is crucial for learning and memory. This may be particularly beneficial in conditions in relation to hearing loss, where neuroplasticity can help the brain adapt to changes in auditory input.

Without intending to be bound by theory, it is believed that, by specifically targeting the CCK-B receptors, the method according to this invention may provide a more focused approach to treatment, potentially reducing side effects compared to less specific treatments, for example, those targeting glutamate receptors. It is also believed that administration of a receptor agonist is generally less invasive than surgery conventionally used to modulate neuroplasticity in brain, making it a more accessible and safer option for patients.

An embodiment of this application relates to a method for treating hearing loss in a subject in need thereof, which includes administrating a CCK-B receptor agonist to the subject.

Without intending to be bound by theory, it is believed the method disclosed herein provides a promising approach to the treatment of hearing-related disease, for example, auditory processing disorder (APD), or the treatment of hearing loss-related cognitive diseases. Furthermore, the drug may facilitate auditory training for patients with cochlear implants or hearing aids. Without intending to be bound by theory, it is believed that CCK-B receptor agonist such as HT-267 can improve hearing sensitivity and improve auditory discrimination ability at behavioral level.

An embodiment of this application relates to a method for improving auditory discrimination ability at behavioral level in a subject in need thereof, which includes administrating a CCK-B receptor agonist to the subject.

Without intending to be bound by theory, it is believed that enhanced auditory discrimination is crucial for language acquisition, music perception and training and is used to help individuals with speech and language disorders. It is believed that CCK-B receptor agonists such as HT-267 can be beneficial in this regard.

### BRIEF DESCRIPTION OF THE DRAWINGS

FIG. 1A is a diagram showing the timeline of the in vivo extracellular recordings experiment (upper), and the representative tuning curve before and after HT-267 administration (lower), according to an embodiment of the present invention;

FIG. 1B shows the normalized fEPSP slope measured 10 minutes before (−10 to 0 min) and 10 minutes after (0-10 min) after injection (0 min) with TF according to an embodiment of the present invention;

FIG. 1C is a diagram showing a comparison of mean normalized fEPSP slope before (−10 to 0 min) and 10 minutes after (0-10 min) in control and HT-267 group according to the embodiment in Fig. B. one-way ANOVA was used to analysis (*p<0.05, **p<0.01);

FIG. 2A is a diagram showing Tone ABR threshold of both ears of mice pre- and 7 weeks post-Tone exposure (TE) according to an embodiment of the present invention: L: exposed left ear; R: Unexposed right ear; Pre-TE(L) vs 7W Post-TE(L), 7W Post-TE(R) vs 7W Post-TE(L), Tukey's multiple comparisons test;

FIG. 2B is a diagram showing Tone ABR threshold of both ears of mice after treatment (HT267/Control) according to an embodiment of the present invention (Multiple paired t tests);

FIG. 2C is a diagram showing differences in ABR thresholds between the post-treated and pre-treated periods in each mouse at different frequencies according to an embodiment of the present invention (Multiple paired t tests);

FIG. 3A shows a schematic diagram of PPI setup according to an embodiment of the present invention;

FIG. 3B is a diagram showing the principle of PPI according to an embodiment of the present invention;

FIG. 3C is a diagram showing the timeline of PPI testing according to an embodiment of the present invention;

FIG. 3D is a chart showing the mean amplitude of acoustic startle response (Startle only trials) at 2k PPI test tasks before and after treatment according to an embodiment of the present invention;

FIG. 3E is a chart showing the PPI ratio (PPI %) in different intensity levels of pre-pulse stimuli (2k) pre- and post-1w_treated (Control/HT-267) according to an embodiment of the present invention;

FIG. 3F is a chart showing the mean amplitude of acoustic startle response (Startle only trials) at 40k PPI test tasks before and after treatment according to an embodiment of the present invention; and

FIG. 3G is a chart showing the PPI ratio (PPI %) in different intensity levels of pre-pulse stimuli (40k) pre- and post-1w_treated (Control/HT-267) according to an embodiment of the present invention.

The figures herein are for illustrative purposes only and are not necessarily drawn to scale.

### DESCRIPTION OF THE PREFERRED EMBODIMENTS

Unless otherwise specifically provided, all tests herein are conducted at standard conditions which include a room and testing temperature of 25 °C, sea level (1 atm.) pressure, pH 7, and all measurements are made in metric units. Furthermore, all percentages, ratios, etc. herein are by weight, unless specifically indicated otherwise. It is understood that unless otherwise specifically noted, the materials compounds, chemicals, etc. described herein are typically commodity items and/or industry-standard items available from a variety of suppliers worldwide.

As used herein, “hearing loss” is defined as diminished acuity to sounds which would otherwise be heard normally.

As used herein, “Auditory Processing Disorder” or “APD” is a hearing loss-related condition caused by something affecting the part of the brain that processes how you hear. APD is a disorder where your brain can't understand and interpret auditory signals the way it should. People with APD can hear, but they may have trouble understanding certain sounds.

As used herein, “presbycusis” or “age-related hearing loss” is hearing loss that occurs gradually for many of us as we grow older.

As used herein, “PPI ratio” refers to the measure of how much a weak sensory stimulus (the prepulse) reduces the reaction to a subsequent strong startling stimulus. This ratio is calculated by comparing the response intensity to the paired weak and strong stimuli with the response to the strong stimulus alone.

An embodiment of this application relates to a method for treating hearing loss in a subject in need thereof, which includes administrating a CCK-B receptor (CCKBR) agonist to the subject with hearing loss.

An embodiment of this application relates to a method for treating auditory processing disorder (APD) in a subject in need thereof, which includes administrating a CCK-B receptor (CCKBR) agonist to the subject.

Without intending to be bound by theory, it is believed that this invention represents a promising approach to the treatment of hearing loss-related diseases for example, auditory processing disorder (APD), through a novel approach combining a pharmacological compound, a CCKBR agonist (such as HT-267), with sound therapy. It is believed that these finding in the present invention imply that CCKBR can serve as a new target with the following functions and applications:

### 1. Treatment of Auditory Processing Disorder (APD).

Without intending to be bound by theory, it is believed that HT-267 could have a potential and positive effect on auditory processing disorder (APD) which is a complex condition affecting the brain's ability to process auditory information. Unlike other forms of hearing loss that are caused by physical damage to the ear, APD originates in the brain, where sound is interpreted. APD can manifest in individuals of any age, though it is most commonly identified in children and the elderly. In elderly, ageing and stroke are the main causes of APD. In children, APD can be associated with complications at the time of birth, such as an early birth or a pregnant person using drugs, or repeated ear infections at a young age. Unfortunately, there is no definitive cure for APD. The common treatment approaches include hearing aids, auditory rehabilitation. The present invention surprisingly finds that HT-267 enhances noise-evoked potentials in the auditory cortex. This suggests HT-267 can improve the ability of the brain to process and interpret sound, which is crucial for understanding speech in a noisy environment. This is further supported by the observed increase in prepulse inhibition (PPI) ratios after treatment, indicating enhanced auditory discrimination in the mice. Without intending to be bound by theory, it is believed that HT-267 can enhance the efficacy of auditory rehabilitation. Overall, HT-267 improves auditory discrimination in mice, it indeed has the potential to be a promising treatment for Auditory Processing Disorder (APD), which involves difficulties in processing auditory information.

### 2. Treatment of Hearing Loss.

Hearing loss, a common condition, affects individuals of all ages. The most prevalent cause is chronic exposure to loud noise, which can damage the sensitive structures within the ear. In elderly, age-related hearing loss, also known as presbycusis, is quite common. Hearing aids and cochlear implants are priority management options for sensorineural hearing loss. Most of ongoing hearing protective drugs are to reduce damage in cochlea hair cells. Here, the present invention proposes another therapeutic strategy that is to target CCKBR in auditory cortex. Without intending to be bound by theory, it is believed that the present invention shows that the application of HT-267 in conjunction with sound therapy in a tone-induced hearing loss mouse model can result in a significant decrease in the threshold of the Auditory Brainstem Response (ABR). It is believed that this reduction in ABR threshold is strong evidence demonstrating that HT-267 may have therapeutic potential to improve sensitivity of the auditory pathway, allowing for better detection of faint sounds. These results are encouraging as this invention suggests a non-invasive method to potentially mitigate hearing loss. The combination of HT-267 and sound therapy could represent a significant advance in the treatment of hearing impairment.

We have previously reported the use of CCKBR agonists in treatment of tinnitus. Reference can be made to Chinese application CN 118304408 A by He et al. to City University of Hong Kong. However, without intending to be bound by theory, it is believed that tinnitus and general hearing loss, or hearing loss-related conditions like auditory processing disorder (APD), involve different underlying mechanisms. Tinnitus is often related to abnormal neural activity in the auditory pathways, while hearing loss can result from damage to the hair cells in the cochlea or other parts of the auditory system. Without intending to be bound by theory, it is believed that the use of CCKBR agonists like HT-267 for tinnitus is based on their ability to modulate neural activity and reduce abnormal signaling. However, the ability of HT-267 to cross the BBB and promote neuroplasticity in the auditory cortex discussed herein has not been reported before. This suggests that HT-267 can directly influence central auditory processing, which is crucial for improving hearing sensitivity and auditory discrimination. This application shows that HT-267 enhances auditory discrimination at the behavioral level, indicating that the drug not only affects physiological aspects of hearing but also translates into functional improvements. This is a critical factor in demonstrating the practical benefits of the treatment. The present invention suggests a broader therapeutic potential of CCKBR agonists.

### 3. Benefits to Hearing Aids or Cochlear Implant Users.

Moreover, it is believed that HT-267 is a potential drug that provides benefits to hearing aids or cochlear implant users by enhancing the effectiveness of the implant and improving hearing outcomes. A cochlear implant enables patient to hear sound. However, this sound is very different from natural hearing. In addition, for some people it may have been a long time since they have heard a sound or the full range of sounds in speech. Therefore, auditory training is necessary for cochlear implant users, which can help patients learn to understand the speech heard through the implant and improve their communication ability. Auditory training should begin within a few weeks of cochlear implantation and lasts for several months. Without intending to be bound by theory, it is believed that HT-267 combined with auditory training may accelerate auditory learning and even improve the user's perception of music and speech in cochlear implant users, which has a lasting impact on their self-confidence and quality of life.

### 4. Treatment of Hearing Loss-Related Cognitive Diseases.

Hearing impairment is recently identified as the most prominent risk factor for dementia. A groundbreaking randomized controlled trial demonstrated that hearing aids could reduce the risk of cognitive decline by nearly 50% over a three-year period in this demographic. This suggests that the treatment of hearing loss via the therapeutic strategy according to this invention could prove beneficial in the treatment of dementia.

### 5. Prevention or Treatment of Parkinson's Disease

A growing body of research has emerged in recent years examining the potential link between hearing loss and Parkinson's disease (PD). Findings suggest that individuals with hearing loss may face a 26% increased risk of developing PD compared to those with normal hearing. Additionally, participants with hearing loss exhibited elevated cumulative morbidity and mortality from PD. The study demonstrated that the utilisation of hearing aids by individuals with hearing impairment was associated with a notable reduction in the prevalence of PD. The incidence of PD exhibited a statistically significant decline over a 10-year period among patients with hearing loss who utilized hearing aids in a timely manner. Hearing loss has been identified as an independent risk factor for the development of PD. It has been demonstrated that even mild hearing loss can have a synergistic effect with the prodromal symptoms of Parkinson's disease, thereby increasing the risk of developing PD. Without intending to be bound by theory, it is therefore believed that patients with mild Parkinson's disease may be able to slow down or even reverse the progression of their condition by using hearing aids in conjunction with CCKBR agonist interventions.

Furthermore, without intending to be bound by theory, it is believed that enhanced auditory discrimination is crucial for language acquisition, music perception and training and is used to help individuals with speech and language disorders. Overall, the implications of HT-267 and development of CCKBR agonist could be far-reaching, offering hope to millions of people suffering from APD, hearing loss around the world.

In an embodiment herein, the CCK-B receptor agonist is HT-267. HT-267 has the following formula (I):

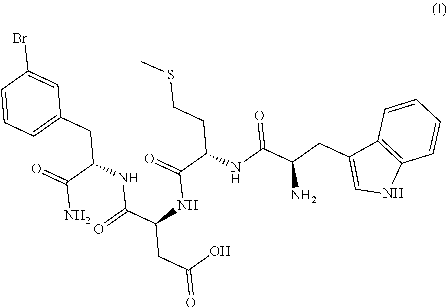 [SMILES: CSCC[C@H](NC(=O)[C@H](N)Cc1c[nH]c2ccccc12)C(=O)N[C@@H](CC(=O)O)C(=O)N[C@@H](Cc1cccc(Br)c1)C(N)=O]

Without intending to be bound by theory, it is believed that CCK plays a significant role in the auditory processing circuitry within the ACx. This application surprisingly found that HT-267 pairing sound exposure can improve hearing sensitivity in mice by increasing noise-evoked potentiation in auditory cortex. After treating mice with HT-267 combined with sound therapy, enhanced prepulse inhibition (PPI) ratios can be observed. Notably, as shown in the examples, the threshold of Auditory Brainstem Response (ABR) decreases in HT-267-treated mice, which demonstrates that HT-267 has a positive effect on the auditory system, reducing hearing damage or enhancing auditory processing. The findings indicate that HT-267 is a promising drug candidate and that CCKBR represents a potential target for the treatment of auditory processing disorder (APD) and hearing loss, as well as for the enhancement of cognitive functions associated with auditory processing.

The first drug to protect hearing from damage caused by cisplatin (a chemotherapy drug), Pedmark, has successfully completed phase III clinical trials and is now approved for use in children undergoing cancer treatment. Pedmark is one formulation of the sodium thiosulfate (STS) which inactivates cisplatin through covalent binding. There are some drugs ongoing clinical trials for the prevention of hearing loss. A randomized, double-blind, placebo-controlled, multicenter Phase 1b clinical trial of DB-020 (a novel formulation of STS) in patients undergoing treatment with cisplatin is now ongoing. These drugs are limited to the treatment of Cisplatin-Induced hearing loss. SENS-401 is the first-in class drug candidate for treatment of sudden sensorineural hearing loss (SSNHL). It is designed to treat or prevent inner ear lesions caused by neurodegeneration and hair cell loss and its phase 1 trial was completed. The limitations of SENS-401 are that treatment initiation needs to be within 96 hours of noise exposure and continued for a prolonged course of up to 28 days. However, this application surprisingly finds that the treatment initiation of HT-267 delays up to 7 weeks post-exposure and it still has a positive effect on hearing improvement.

Without intending to be bound by theory, it is also believed that CCKBR agonist like HT-267 can enhance hearing sensitivity in patients with partial hearing impairment. On the other hand, in patients with cochlear implants, the combination of HT-267 during the auditory training phase can improve the patients' auditory learning ability.

In an embodiment herein, the CCK-B receptor agonist is administrated once daily for a week. Without intending to be bound by theory, it is believed that once-daily administration ensures that the CCK-B receptors are consistently activated, which can be crucial for achieving the desired therapeutic effects, such as improved hearing sensitivity.

In an embodiment herein, the CCK-B receptor agonist is administrated at a dosage of from about 0.1 mg/kg to about 5 mg/kg. For example, the agonist can be administrated at a dosage of about 0.2 mg/Kg, about 0.5 mg/Kg, about 1 mg/Kg, about 2 mg/Kg, about 3 mg/Kg, or about 4 mg/Kg. Without intending to be bound by theory, it is believed that the dosage in the range mentioned above, in particular the dosage of about 0.2 mg/Kg, is beneficial for hearing sensitivity.

In an embodiment herein, the administrating step comprises a route selected from the group consisting of oral administration, intraperitoneal administration (i.p.), intravenous administration, and a combination thereof. In some embodiments, the CCK-B receptor agonist is administrated intraperitoneally. Without intending to be bound by theory, it is believed that intraperitoneal administration allows for rapid absorption of the drug into the bloodstream, leading to a quicker onset of effects compared to other routes like oral administration. In addition, drugs administered intraperitoneally bypass the liver initially, avoiding first-pass metabolism and potentially increasing the bioavailability of the drug.

In some embodiments herein, the CCK-B receptor agonist herein can be manufactured as a medicament in a dosage form suitable for oral administration, intraperitoneal injection or intravenous administration. For example, the medicament may be a tablet or an injection.

In some embodiments herein, CCK-B receptor agonists, such as HT-267, are included in a medicament in an amount of 0.02% by weight.

In an embodiment herein, the method further comprises subjecting the subject to a sound exposure after administrating the CCK-B receptor agonist.

In an embodiment herein, the sound exposure can be performed at a frequency ranging from about 2 kHz to about 44 kHz. In an embodiment herein, the sound exposure is performed with tone ranging from 9 kHz to about 28.7 kHz and white noise which has equal intensity.

In an embodiment herein, the sound exposure includes about 300 to about 1000 trials. In some embodiments, each of the trials lasts about 1000 ms consisting of about 100 to 200 ms pulse and about 800 ms to about 900 ms inter-trial interval. Without intending to be bound by theory, it is believed that this number of tests is not too high to cause stress to the subject, nor too low to induce synaptic plasticity.

In an embodiment herein, the CCK-B receptor agonist can improves hearing sensitivity in the subject.

In an embodiment herein, the CCK-B receptor agonist improve auditory discrimination ability at behavioral level in the subject.

These above described aspects of the method for treating hearing loss according to this invention also apply to the other methods herein, unless otherwise specified.

In some embodiments, the method can be used for treating a subject suffering from hearing loss and a cognitive disease that may be in connection with the hearing loss. For example, the cognitive disease may be Parkinson's disease. Without intending to be bound by theory, it is believed that CCK-B receptor agonists may also have cognitive benefits, such as improving memory and learning in conditions such as Alzheimer's disease. This could be an added benefit for patients with hearing loss, who may also experience cognitive decline. Without intending to be bound by theory, it is believed that that the method herein can slow down or even reverse the progression of hearing-loss related cognitive diseases (such as mild Parkinson's disease) in patients by using hearing aids in conjunction with CCKBR agonist interventions.

An embodiment of this application relates to a method for improving neuroplasticity in the auditory cortex of a subject in need thereof, which includes administrating a CCK-B receptor agonist to the subject.

In some embodiments, the CCK-B receptor agonist is HT-267. In some embodiments, the CCK-B receptor agonist is administrated at a dosage from about 0.1 mg/kg to about 5 mg/kg, for example, about 0.2 mg/Kg, about 0.5 mg/Kg, about 1 mg/Kg, about 2 mg/Kg, about 3 mg/Kg, or about 4 mg/Kg. In some embodiments, the administrating step includes a route selected from the group consisting of oral administration, intraperitoneal administration, intravenous administration, and a combination thereof. In particular, the agonist is administrated by intraperitoneal administration. In some embodiments, the method further includes subjecting the subject to a sound exposure after administrating the CCK-B receptor agonist. In some embodiments, the sound exposure is performed at a frequency ranging from about 2 kHz to about 44 kHz. In some embodiments, the sound exposure includes about 500 trials. For example, each of the trials lasts about 1000 ms consisting of about 100 ms pulse and 900 ms inter-trial interval. Other aspects and benefits of this method are described above.

An embodiment of this application relates to method for improving auditory discrimination ability at behavioral level in a subject in need thereof, which includes administrating a CCK-B receptor agonist to the subject.

In some embodiments, the CCK-B receptor agonist is HT-267. In some embodiments, the CCK-B receptor agonist is administrated daily for a week. In some embodiments, the CCK-B receptor agonist is administrated at a dosage of from about 0.1 mg/kg to about 5 mg/kg, for example, about 0.2 mg/Kg, about 0.5 mg/Kg, about 1 mg/Kg, about 2 mg/Kg, about 3 mg/Kg, or about 4 mg/Kg. In some embodiments, the administrating step includes a route selected from the group consisting of oral administration, intraperitoneal administration, intravenous administration, and a combination thereof. In particular, the agonist is administrated by intraperitoneal administration. In some embodiments, the method further includes subjecting the subject to a sound exposure after administrating the CCK-B receptor agonist, wherein the sound exposure is performed at a frequency ranging from about 2 kHz to about 40 kHz. In some embodiments, the sound exposure includes about 300 to about 1000 trials. In particular, each of the trials lasts about 1000 ms consisting of about 200 ms pulse and 800 ms inter-trial interval. Other aspects and benefits of this method are described above.

An embodiment of this application relates to a method for improving hearing sensitivity in a subject in need thereof, which includes administrating a CCK-B receptor agonist to the subject.

An embodiment of this application relates to a method for improving auditory discrimination ability at behavioral level in a subject.

An embodiment of this application relates to use of a CCK-B receptor agonist for treating auditory processing disorders/improving hearing sensitivity/improving auditory discrimination ability at behavioral level in a subject in need thereof.

An embodiment of this application relates use of a CCK-B receptor agonist in the manufacture of a medicament for treating auditory processing disorders/improving hearing sensitivity/improving auditory discrimination ability at behavioral level in a subject in need thereof.

### EXAMPLE

### Methods

### 1. Animal

F1 offspring from C57BL and CBA/ca (HKU) breed at 12 h light (8 P.M-8 A.M)-12 h (8 A.M-8 P.M) dark room. All mice are provided food and water ad libitum. Cages are cleaned weekly. All experimental procedures are approved by the Animal Subjects Ethics Subcommittees of the City University of Hong Kong.

### 2. In Vivo Extracellular Recordings in Anesthetized Mice

Mice are anesthetized using sodium urethane (1.8 g/kg, i.p.). And anesthetics are replenished periodically throughout the surgery and electrophysiological recording. 30 minutes before induction of anesthesia, mice are given dexamethasone (5 mg/kg, i.p.) to prevent cerebral edema. Also to minimize suffering, atropine sulfate (0.05 mg/kg, s.c.) is injected 15 minutes before induction of anesthesia. Animals are fixed on a stereotaxic frame (Narishige, Japan) and body temperature is maintained between 37-38 °C using a feedback-controlled heating pad (RWD, China). After the surface of the skull is thoroughly cleaned, the muscles and skull covering the auditory cortex are removed. Silicone oil is applied to keep the exposed brain tissue moist. A homemade recording 4-electrode array (~1.0 M (2, FHC) is inserted into layer IV of the auditory cortex. The auditory stimuli including 100 noise bursts ((Intensity, 70 dB; Duration, 100 ms; Inter-stimulus-interval, 10 s), and a series of tones (frequency: 2750, 3158.92, 3628.65, 4168.22, 4788.03, 5500, 6317.84, 7257.29, 8336.44, 9576.06, 11000, 12635.7, 14514.6, 16672.9, 19152.1, 22000, 25271.4, 29029.2, 33345.8, 38304.2, and 44000 Hz, intensity: 60 to 90 dB SPL, 5 dB/increment) are generated by a TDT workstation (TDT Inc., USA). Extracellular signals and local field potential (LFP) signals are also recorded by the TDT workstation. When the depth of each tetrode is determined, Tunning curves are collected. There are total 147 Frequency×Amplitude pairs in auditory stimuli. For each pair 5 repeats are done during each tuning curve generation, resulting in 735 trials, each lasting 0.5 seconds. 50 ms tones are presented at the beginning of each trial. The Frequency×Amplitude pair list is pseudo-randomly shuffled at the beginning of the experiment. Analysis of the data is performed using custom MATLAB scripts.

### 3. Tone Exposure (TE) Induced Unilateral Hearing Loss

The mice are anesthetized via an intraperitoneal injection of pentobarbital (35 mg/kg, i.p.). A sound-attenuating foam is then introduced into the right ear canal. Subsequently, the animals are placed individually on a pad in front of a speaker that delivered a constant, one-hour pure 12.6 kHz tone calibrated at 116 dB SPL. The left ear, which is exposed, is oriented in direct alignment with the speaker. To prevent movement during the experiment, the mice are attached to the pad with removable tape. Following a period of 60 minutes, the animal is removed from the exposure chamber and transferred to a heating pad to facilitate recuperation from anesthesia. The calibration of the sound is performed with the aid of a microphone (BK) and conditioning amplifier (BK), in conjunction with bespoke MATLAB and TDT scripts.

### 4. Auditory Brainstem Response (ABR)

The protocol reference for ABR Testing is from the ABR User Guide (Tucker-Davis Technologies, Inc. (TDT)). The RZ6-A-P1 Multi I/O Processor can generate a digital stimulus, which are converted into acoustic stimuli via TDT's MF1 Multi-Field Magnetic Speaker. Anesthetized mice are tested in Closed Field experiments mode. Biological signals are digitized on a Medusa4Z preamplifier and transferred to the RZ6. BioSigRZ software provides a simplified interface for selecting stimuli and configuring how biological responses are recorded and processed.

For Tone ABRs, Default file Root_ToneAbr.acf from TDT are modified and used. A five millisecond, single-channel cosine-squared gated tone will be presented 21 times per second at the following frequencies: 4 kHz, 8 kHz, 12 kHz, 16 kHz, 20 kHz, 24 kHz, 28 kHz and 32 kHz. For each frequency, the tone level will vary from 90 dB SPL to 20 dB SPL in 5 dB decrements. With each successive presentation, the tone phase will alternate 180 degrees to eliminate potential speaker artifacts. A 10 ms response window is acquired from a single acquisition channel on the preamplifier via the RZ6 fiber optic port. A total of 512 responses will be averaged at each frequency and level combination. ABR Waveforms are analyzed by custom-coding MATLAB script.

### 5. Therapy

For acute electrophysiological experiments in Example 1, the mouse is intraperitoneally administrated with HT-267 (0.5 mg/kg) or the volume of vehicle solution, meanwhile, the speaker played target frequency tone (6k Hz) ((100 ms pulse+900 ms ITI)/trial, 500 trials total) which is generated and control by TDT station.

For chronic ABR test experiment in Example 2, the same treatment protocol as in our previous patent (CN 118304408 A) is used for hearing loss mice. The HT-267 compounds cannot be purchased on the market, and those used herein are synthesized exclusively in the lab of Dr. Micky Tortorella who has the patent for the HT-267 compound. The purity of HT-267 powder should be >95% (HPLC). HT-267 power is dissolved in Dimethyl sulfoxide (DMSO) with the stock concentration of 10 mM. The drug usually is stored at −20 °C. On the day of injection, the stock solution is diluted with saline freshly to the injection concentration—320 μM, including 3.2% DMSO and 2% Cremophor EL. In this case, the treatment course includes 4 treatment cycles, treatment given for one day followed by one day of rest is one treatment cycle.

For the behavioral experiment in Example 3, animal is intraperitoneally administrated with HT-267 (0.2 mg/kg) or vehicle pairing with 2k Hz tone ((200 ms pulse+800 ms ITI)/trial, 1000 trials total) daily for one week.

### 6. Pre-Pulse Inhibition (PPI) Test

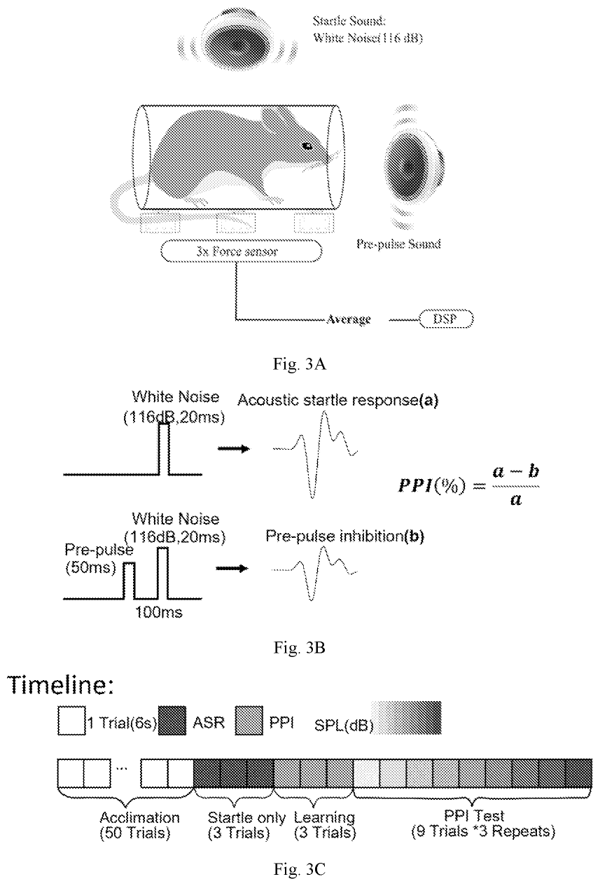

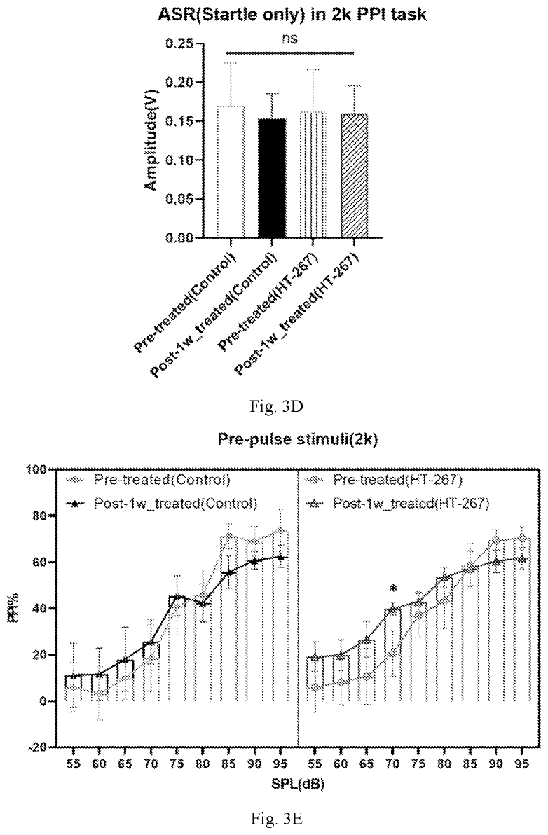

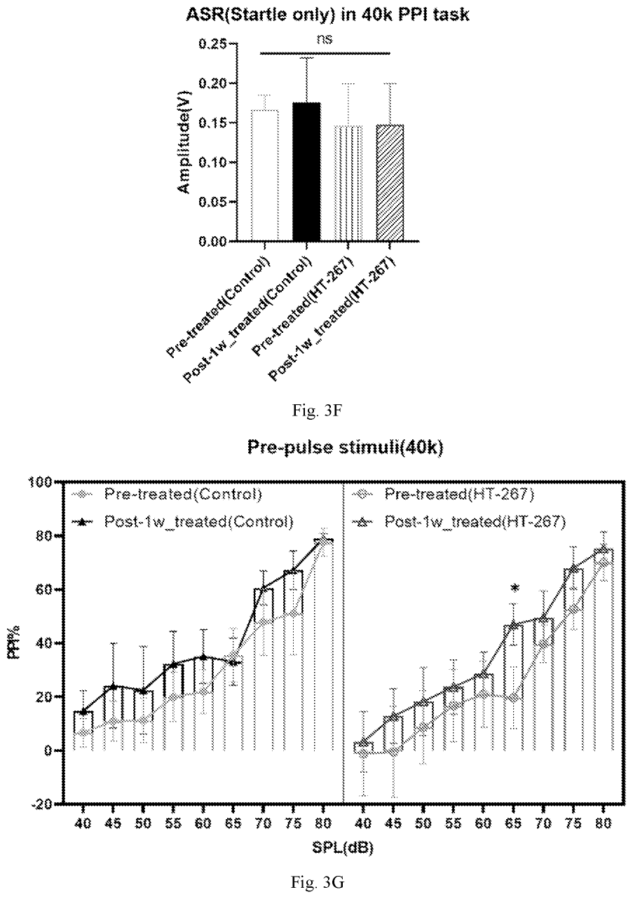

Two pure tones with different frequencies (2 kHz and 40 kHz) are selected as acoustic probe stimuli, and the pre-pulse inhibition (PPI) to each tone is measured under different intensities (FIG. 3). The working principle of PPI is shown in FIG. 3A. A loud white noise (116 dB, 20 ms) is used to induce startle responses. However, a pre-pulse sound (50 ms) 100 ms before the loud sound inhibits the startle reflex, namely pre-pulse inhibition (PPI). The procedure of PPI comprises four sections, including the acclimation, acoustic startle, learning, and PPI test. Acclimation lasted 5 minutes, followed by 3 startle trials to stabilize startle response and 3 PPI trials for mouse learning (loudest pre-pulse auditory stimuli in PPI test section) after acoustic startle trials. Next, 18 trials of PPI experiments are carried out. The shade of red indicates the intensity of the pre-pulse sound pressure level (SPL). Each square represents 6 seconds. The PPI in startle response for 2 kHz and 40 kHz pure tone under different pre-pulse sound intensities (2k: 55-95 dB, in 5 dB increments; 40k: 40-80 dB, in 5 dB increments) are measured. PPI is calculated as a percentage score: PPI (%)=(startle response for pulse alone-startle response for pulse with pre-pulse)/startle response for pulse alone)×100.

### Example 1

### HT-267 Crossing Blood-Brain Barrier Enables Neuroplasticity in the Auditory Cortex

It is known that HT-267 can mimic the role of CCK and facilitate the necessary synaptic changes for LTP induction by specifically targeting CCKBR (He et al., CN 118304408 A). To investigate neuroplastic changes in the auditory cortex after pharmacological administration of HT-267 to animals, an acute experiment is performed, in which recording electrodes are implanted in the primary auditory cortex to observe and record spike (action potential) signals and local field potentials.

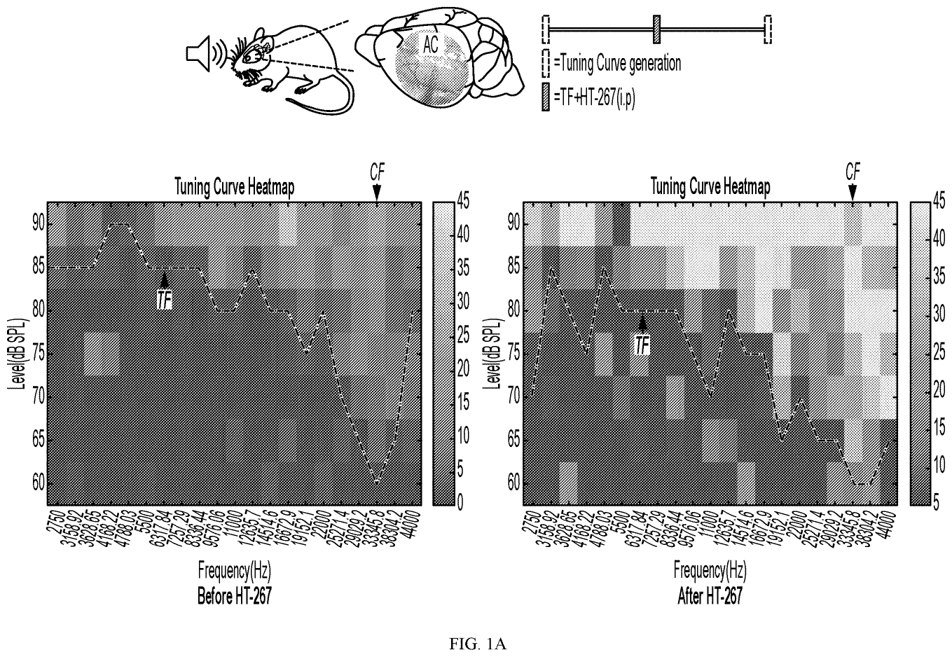

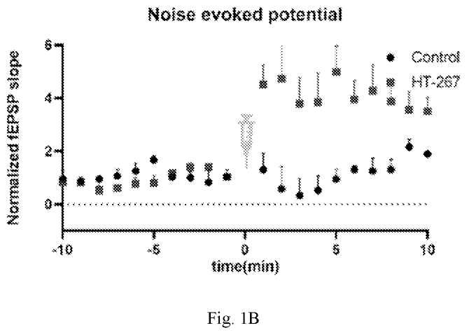

The timeline of the acute experiment is shown in FIG. 1A (top). The whole procedure is divided into three stages. HT-267 paired tones are given separately and two tuning curves are generated to measure the plasticity changes before and after treatment. The characteristic frequency (CF) of the primary recording site is determined by constructing a tuning curve. A tuning curve is a graph that describes the response of a neuron or sensory receptor to stimulation at different frequencies. The tuning curve allows the determination of the frequency at which the neuron exhibits the highest or most sensitive response, which corresponds to the characteristic frequency of the neuron. The target frequency (TF) is always determined and selected at the edge of the tuning curve at a specific site. In this example, we intraperitoneally inject HT-267 (0.2 mg/kg, ip) and expose the animals to a pure tone of 6 kHz. Representative results show that the tuning curves shifted significantly and broadened after treatment, especially near the 6 kHz frequency (FIG. 1A, lower panel). Notably, there is a significant increase in the number of spikes of auditory cortex neurons responding to tones at different frequencies and sound pressures, especially at frequencies above 6 kHz. Next, Noise-evoked the field excitatory postsynaptic potential (fEPSP) is also measured before and after injection pairing with TF. Compared to the control group, Noise-evoked fEPSP slope in HT-267 group significantly increased (FIG. 1B), which indicates the increased activity of the auditory cortex in response to noise. This result supports that HT-267 crossing Blood-Brain Barrier enables neuroplasticity in the auditory cortex, and HT-267 exhibits ability to enhance synaptic plasticity and strengthen synaptic connections. It is believed that the potential applications of HT-267 may be used to treat hearing impairments and enhance cognitive functions related to auditory processing, such as improving attention related to sound.

As shown in FIGS. 1A to 1C, HT-267 that crossed Blood-Brain Barrier (BBB) enables neural plasticity in the auditory cortex. FIG. 1A shows the timeline of the in vivo extracellular recordings experiment. Two tuning curves are generated throughout the acute experiment. Recording electrodes are placed in layer IV of auditory cortex. After generating tuning curve, the characteristic frequency (CF) and shape of tuning curve is determined for each recording site and target frequency (TF) is determined (TF is always chosen at the edge of the tuning curve). TF is immediately played for 10 minutes after HT-267 is injected (i.p). Then, one more tuning curve is generated. The lower panels of FIG. 1A show the representative tuning curve before and after HT-267 administration. The tuning curve is enlarged in the 6k Hz range after HT-267 Intraperitoneal administration & target tone pairing (indicated by the arrow with TF). FIG. 1B shows the normalized fEPSP slope measured 10 minutes before (−10 to 0 min) and 10 minutes after (0-10 min) after injection (0 min) with HT-267. FIG. 1C shows the comparison of mean normalized fEPSP slope before (−10 to 0 min) and 10 minutes after (0-10 min) in control and HT-267 group. One-way ANOVA is used to analysis (*p<0.05, **p<0.01).

### Example 2

### HT-267 Improves Hearing Sensitivity in Tone-Induced Hearing Loss Mice

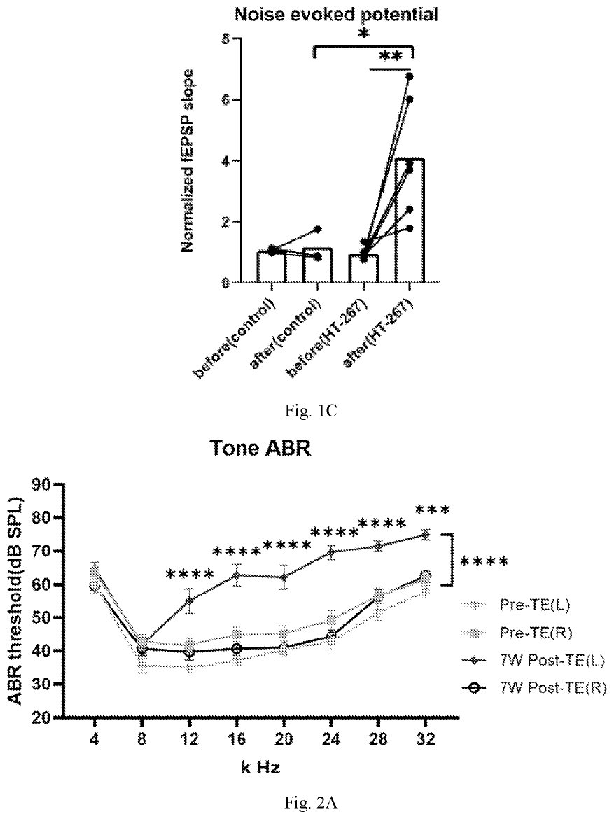

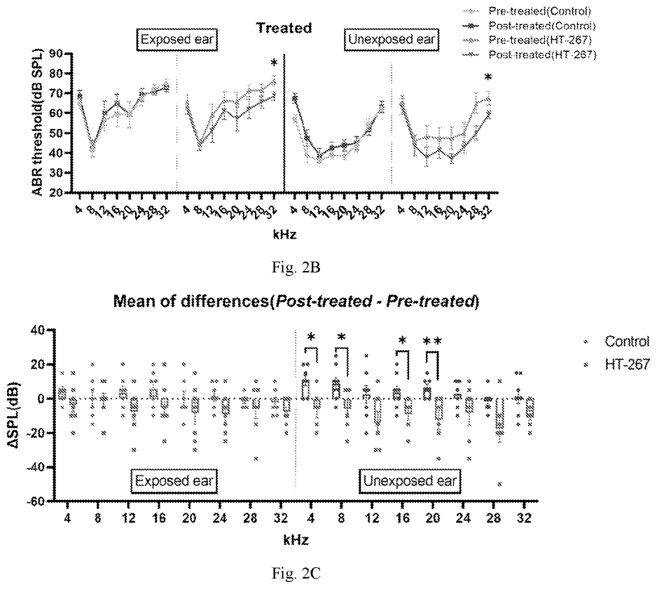

To prove that HT-267 could be used to treat hearing loss, this example tests how HT-267 affects hearing sensitivity in hearing-impaired mice. Firstly, to verify the effectiveness of a one-hour exposure to a 12.6 kHz pure tone in inducing hearing loss. Auditory brainstem responses (ABRs) are measured in both ears 7 weeks after the tone exposure. The ABR test measures the electrophysiologic activity generated when ear is exposed to auditory stimuli. The ABR test can provide information about the sensitivity of the auditory system, the presence of hearing loss, and the overall function of the auditory pathway. The results show that the ABR thresholds of the exposed ear are significantly elevated above 12 kHz compared to the same ear before the tone exposure, as well as compared to the unexposed ear (FIG. 2A). The elevated ABR thresholds indicate that the mice developed hearing loss in the frequency range affected by the tone exposure.

Next, mice shown hearing loss are given treatment 7 weeks post Tone exposure. Tone ABR thresholds are also measured after treatment. Notably, in HT-267 group, a decrease in tone ABR thresholds is observed in both ears in hearing loss mice, mice in the control group fail to show reduced tone ABR thresholds (FIG. 2B). These results suggest an improvement in hearing sensitivity. Furthermore, the alterations in ABR thresholds observed in each mouse before and after the administration of the treatment are subjected to a statistical analysis (FIG. 2C). The majority of the differences in ABR thresholds between the post-treated and pre-treated periods in the HT-267 group are negative, indicating a decrease in thresholds. In contrast, the majority of the differences in the control group are positive, indicating an increase in thresholds. These imply that the mice in the control group continue to experience hearing loss, whereas HT-267 prevents further hearing loss and even exhibits an improvement in hearing in both ears of mice with hearing loss.

FIG. 2A shows the tone ABR threshold of both ears of mice pre- and 7 weeks post-Tone exposure (TE). L: exposed left ear. R: Unexposed right ear. Pre-TE(L) vs 7W Post-TE(L), 7W Post-TE(R) vs 7W Post-TE(L), Tukey's multiple comparisons test. FIG. 2B shows the tone ABR threshold of both ears of mice after treatment (HT267/Control). Multiple paired t tests. FIG. 2C shows the differences in ABR thresholds between the post-treated and pre-treated periods (ABR threshold of Post-treated minus ABR threshold of Pre-treated) in each mouse at different frequencies. Multiple unpaired t tests. All data are presented as mean±SEM, *P≤0.05, **P≤0.01, ***P≤0.001. ****P≤0.0001. The results in FIGS. 2A-2C show that HT-267 can improve hearing sensitivity in Tone-induced hearing loss mice.

### Example 3

HT-267 could Improve the Auditory Discrimination Ability at the Behavioral Level

This example is to investigate the functional effects of HT-267-induced LTP on auditory cortex. To determine whether this enhanced synaptic plasticity translates into improved auditory perception, this example designs auditory discrimination task-Pre-pulse inhibition (PPI) test (FIG. 3A). A set of pre-pulse auditory stimuli with different characteristics is generated. These stimuli may include different tones (2k or 40k) of different intensity levels.

Mice are tested in both 2k and 40k PPI tasks before treatment. Following 1-week treatment (administration of Control/HT-267 pairing with 2k tone), mice are subjected to 2k and 40k PPI tasks again. It is found that the mean amplitude of acoustic startle response (Startle only trials) show no significant change before and after treatment, which means LTP induced by HT-267 has no significant effect on the overall magnitude of the startle response (FIGS. 3D and 3F). This suggests that HT-267 may not have directly influenced the neural circuits (Cochlea→the caudal nucleus of the pontine reticular formation (PnC)) for generating the startle response. However, the PPI ratio of the 2k (70 dB) stimulus significantly enhances after HT-267 application, while the PPI ratios at other intensity levels showed no significant change (FIG. 3E). Interestingly, improved performance in the 40k PPI test in mice injected with HT-267 is observed (FIG. 3G). The result is consistent with previous electrophysiological findings that neurons in the auditory cortex increase their activity in response to tones not only at the target frequency but also at other frequencies after HT-267 injection (FIG. 1A), which implies that HT-267 pairing with targeted tone positively affects auditory discrimination.

FIG. 3A shows a schematic diagram of PPI setup. Startle sound speaker and pre-pulse sound speaker are placed beside the mouse. Signals from three force sensors placed under the testing tube are averaged in a custom-made circuit and transmitted to digital signal processor (DSP) for further analysis. FIG. 3B is a diagram showing the principle of PPI. Mouse is startled when playing a loud white noise (116 dB, 20 ms) called Acoustic startle response (ASR). However, a pre-pulse sound (50 ms) 100 ms before startle sound will inhibit startle reflex, which is called pre-pulse inhibition (PPI). FIG. 3C shows the timeline of PPI testing. Testing compromises of 4 sections. Acclimation lasts 5 minutes, followed by 3 ASR trials to stabilize startle response and 3 PPI trials for mouse learning after ASR trials. Next, 27 trials PPI is followed. The shade in the blocks of the figure indicates the intensity of pre-pulse sound pressure level (SPL). Each square represents 6 seconds. FIG. 3D is a chart showing the mean amplitude of acoustic startle response (Startle only trials) at 2k PPI test tasks before and after treatment. FIG. 3E shows the PPI ratio (PPI %) in different intensity levels of pre-pulse stimuli (2k) pre- and post-1w_treated (Control/HT-267). Superimposed symbols/bars (Mean) with error bars (SEM) are plotted, Multiple paired t test is used to analysis (*p<0.05). FIG. 3F is a chart showing the mean amplitude of acoustic startle response (Startle only trials) at 40k PPI test tasks before and after treatment. FIG. 3G shows the PPI ratio (PPI %) in different intensity levels of pre-pulse stimuli (40k) pre- and post-1w_treated (Control/HT-267). Superimposed symbols/bars (Mean) with error bars (SEM) are plotted, Multiple paired t test is used to analysis (*p<0.05). The results in FIGS. 3A-3G shows that HT-267 can improve the auditory discrimination ability at the behavioral level.

It should be understood that the above only illustrates and describes examples whereby the present invention may be carried out, and that modifications and/or alterations may be made thereto without departing from the spirit of the invention.

It should also be understood that certain features of the invention, which are, for clarity, described in the context of separate embodiments, may also be provided in combination in a single embodiment. Conversely, various features of the invention which are, for brevity, described in the context of a single embodiment, may also be provided separately, or in any suitable subcombination.

All references specifically cited herein are hereby incorporated by reference in their entireties. However, the citation or incorporation of such a reference is not necessarily an admission as to its appropriateness, citability, and/or availability as prior art to/against the present invention.

What is claimed is:

1. A method for improving neuroplasticity in the auditory cortex in a subject in need thereof, comprising administrating a CCK-B receptor agonist to the subject.

2. The method of claim 1, wherein the CCK-B receptor agonist is HT-267.

3. The method of claim 1, wherein the CCK-B receptor agonist is administrated at a dosage from about 0.1 mg/kg to about 5 mg/kg.

4. The method of claim 1, wherein the administrating step comprises a route selected from the group consisting of oral administration, intraperitoneal administration, intravenous administration, and a combination thereof.

5. The method of claim 1, further comprising subjecting the subject to a sound exposure after administrating the CCK-B receptor agonist.

6. The method of claim 5, wherein the sound exposure is performed at a frequency ranging from about 2 kHz to about 44 kHz.

7. The method of claim 5, wherein the sound exposure includes about 500 trials.

8. The method of claim 5, wherein each of the trials is about 1000 ms consisting of about 100 ms pulse and 900 ms inter-trial interval.

9. A method for treating hearing loss in a subject in need thereof, comprising administrating a CCK-B receptor agonist to the subject.

10. The method of claim 9, wherein the CCK-B receptor agonist is HT-267.

11. The method of claim 9, wherein the CCK-B receptor agonist is administrated for 4 treatment cycles, each cycle comprising one day treatment followed by one day of rest.

12. The method of claim 9, wherein the CCK-B receptor agonist is administrated at a dosage of from about 0.1 mg/kg to about 5 mg/kg.

13. The method of claim 9, wherein the administrating step comprises a route selected from the group consisting of oral administration, intraperitoneal administration, intravenous administration, and a combination thereof.

14. The method of claim 9, further comprising subjecting the subject to a sound exposure after administrating the CCK-B receptor agonist.

15. The method of claim 14, wherein the sound exposure includes white noise and 5 tone frequencies ranging from 9 kHz to about 28.7 kHz.

16. The method of claim 14, wherein the sound exposure includes about 300 to about 1000 trials.

17. The method of claim 14, wherein each trial lasts 1,400 milliseconds and includes 100 milliseconds of white noise commencing at 150 milliseconds and 600 milliseconds, as well as 100 milliseconds of five merged frequencies beginning at 250 milliseconds and 500 milliseconds.

18. The method of claim 9, wherein the CCK-B receptor agonist improves hearing sensitivity in the subject.

19. A method for improving auditory discrimination ability at behavioral level in a subject in need thereof, comprising administrating a CCK-B receptor agonist to the subject.

20. The method of claim 19, wherein the CCK-B receptor agonist is HT-267.

21. The method of claim 19, wherein the CCK-B receptor agonist is administrated daily for a week.

22. The method of claim 19, wherein the CCK-B receptor agonist is administrated at a dosage of from about 0.1 mg/kg to about 5 mg/kg.

23. The method of claim 19, wherein the administrating step comprises a route selected from the group consisting of oral administration, intraperitoneal administration, intravenous administration, and a combination thereof.

24. The method of claim 19, further comprising subjecting the subject to a sound exposure after administrating the CCK-B receptor agonist, wherein the sound exposure is performed at a frequency ranging from about 2 kHz to about 40 KHz.

25. The method of claim 19, wherein the sound exposure includes about 300 to about 1000 trials.

26. The method of claim 19, wherein each of the trials is about 1000 ms consisting of about 200 ms pulse and 800 ms inter-trial interval.
